(19) United States
(12) Patent Application Publication
(10) Pub. No.: US 2017/0065825 A1
(43) Pub. Date: Mar. 9, 2017

(54) APPARATUS AND METHOD FOR IMAGE-GUIDED TEMPORARY DENERVATION

(71) Applicant: WEINBERG MEDICAL PHYSICS LLC, North Bethesda, MD (US)

(72) Inventor: Irving Weinberg, North Bethesda, MD (US)

(73) Assignee: Weinberg Medical Physics LLC

(21) Appl. No.: 15/260,453

(22) Filed: Sep. 9, 2016

Related U.S. Application Data
(60) Provisional application No. 62/216,048, filed on Sep. 9, 2015.

(51) Int. Cl. A61N 2/00; A61B 5/00; A61N 2/02; A61B 5/055

(52) U.S. Cl. CPC A61N 2/006; A61B 5/055; A61B 5/4836; A61N 2/02; A61B 5/4824

(57) ABSTRACT

Disclosed embodiments are directed to a method and apparatus for production of pulsed magnetic fields that are focused on one or more innervated pain assemblies and improve the ability of a surgeon or diagnostician to select an appropriate target area for intervention, and to assist in determining whether an intervention will be successful in alleviating symptoms.

### CROSS REFERENCE

This application claims priority under 35 U.S.C. 119(e) to U.S. Provisional Patent Application No. 62/216,048 (incorporated by reference in its entirety) filed on Sep. 9, 2015, entitled “APPARATUS AND METHOD FOR IMAGE-GUIDED TEMPORARY DENERVATION.”

### FIELD

Disclosed embodiments are directed to a method and apparatus for production of pulsed magnetic fields that are focused on one or more innervated pain assemblies.

### SUMMARY

Disclosed embodiments are directed to a method and apparatus that improve the ability of a surgeon or diagnostician to select an appropriate target area for intervention, and to assist in determining whether an intervention will be successful in alleviating symptoms.

In accordance with at least one disclosed embodiment, an apparatus and associated methodologies are provided that utilize one or more electromagnetic coils activated by an electrical generator that produce pulsed magnetic fields that are focused on one or more innervated pain assemblies suspected of causing pain, and one or more electromagnetic coils producing pulsed magnetic fields for imaging the region that includes the one or more nerve or innervated pain assemblies.

In accordance with at least one disclosed embodiment, the nerve-affecting coils may be segmented, with sections that may be selectively energized, de-energized, or otherwise modulated in order to move the location of maximal magnetic field.

### BRIEF DESCRIPTION OF THE FIGURES

The detailed description particularly refers to the accompanying figures in which:

FIG. 1 provides a schematic of apparatus components utilized in accordance with the disclosed embodiments shown adjacent to the spine

### DETAILED DESCRIPTION

Magnetostimulation of portions of the human nervous system (or that of other animals) has been promoted for various purposes. Transcranial stimulation at low frequencies (e.g., around 1 Hz) has been shown to be effective in reducing seizure frequency. Pulsed transcranial magnetic stimulation (delivered in about a millisecond) has been shown effective in reducing migraine attacks. Magnetic stimulation of peripheral nerves has been used diagnostically, in order to determine the threshold for excitation and thereby assess for peripheral neuropathy.

Transcranial stimulation has been promoted for treatment of chronic pain, as reported by Julien Nizard in Jul. 24, 2012 article entitled “Non-invasive Stimulation Therapies for the Treatment of Refractory Pain,” published in the journal Discovery Medicine and incorporated herein by reference in its entirety. A survey of magnetic and electrical applications for stimulation and inhibition of nerves was published in 2015 by P. M. Rossini et al, in the journal Clinical Neurophysiology, volume 126, pages 1071-1107, and is incorporated herein by reference in its entirety. Electrical pulses can be applied to peripheral nerves (transcutaneous electrical nerve stimulation, or “TENS”) in order to rapidly relieve pain, as taught by J. M. DeSantana et al, in the 2008 article published in the journal Current Rheumatology, entitled “Effectiveness of Transcutaneous Electrical Nerve Stimulation in Treatment of Hyperalgesia and Pain,” incorporated herein by reference in its entirety.

Additionally, it is known that surgery or interventional procedures such as epidural steroid injections can be helpful in reducing pain caused by neural compression, or in reducing pain transmitted from painful structures such as herniated discs or facet joint capsules.

Spinal surgeons generally request that imaging studies be performed prior to performing an interventional procedure, in order to assist in determining the location of the nerve activation that is responsible for the pain. The most common study ordered is Magnetic Resonance Imaging (MRI), which is often unreliable in predicting the location of the cause of pain, as documented by T. Maus in the 2010 review article entitled “Imaging the back pain patient”, published in the journal Physical Medicine and Rehabilitation Clinics of North America, Volume 21, number 4, pages 725-66, and incorporated herein by reference in its entirety.

With this understanding of the conventional art in mind, disclosed embodiments are intended to improve the ability of a surgeon or diagnostician to select an appropriate target for intervention, and to assist in determining whether an intervention will be successful in alleviating symptoms. For the purpose of describing disclosed embodiments, the term “innervated pain assembly” is intended to include one or more portions of nerves, nerve roots, ganglia, and/or nerve bundles, whether the nerves are of afferent or efferent types, and may include one or more innervated tissue elements such as muscle, bone, disc, skin or joint capsule. Thus, an innervated pain assembly may be located, for example, in or near a subject's spine, or may be a dermatome. A volume or area containing the innervated pain assembly and nearby tissues is termed the “region of interest”.

Disclosed embodiments provide an apparatus that utilizes one or more electromagnetic coils activated by an electrical generator (“nerve-affecting coils”) that produce pulsed magnetic fields that are focused on one or more innervated pain assemblies suspected of causing pain, and one or more electromagnetic coils producing pulsed magnetic fields for imaging the region that includes the one or more nerve or innervated pain assemblies (“imaging coils”).

Optionally, the nerve-affecting coils may be segmented, with sections that may be selectively energized, de-energized, or otherwise modulated in order to move the location of maximal magnetic field. This selective energizing process is similar in principle to the concept of shimming in a magnetic resonance field.

It is understood that the nerve-affecting coil, which may include multiple coils that may be activated independently, may be optimized to deliver magnetic pulses to a region as small as possible, for example, following the methods of David Cohen and B. Neil Cuffin in their survey article entitled “Developing a More Focal Magnetic Stimulator,” published in the 1991 Journal of Clinical Neurophysiology, volume 8, section 1, pages 102-111, and incorporated herein by reference in its entirety. It is understood that mathematical optimization routines may be employed to design and activate a nerve-affecting coil, for example, following the examples used in MRI design and summarized by Peter T. While, Larry K. Forbes, and Stuart Crozier, in the article entitled “3D Gradient Coil Design—toroidal surfaces,” published in the Journal of Magnetic Resonance in 2008 (volume 198, pages 31-40), incorporated herein by reference in its entirety.

The term “electromagnetic generator” is intended to include batteries or other electrical supplies whose outputs are modulated by one or more switches or circuits. The term “imaging coils” is used to include one or more of the following: radiofrequency coils (for transmission and/or receipt of radiofrequency energy), magnetic polarization and/or pre-polarization coils, and magnetic gradient coils.

In accordance with at least one disclosed embodiment, it should be understood that one or more of the nerve-affecting coils may also be one or more of the imaging coils, and vice versa.

In accordance with at least one disclosed embodiment, the imaging and/or nerve-affecting coils may be arrayed in planar and/or single-sided form in order to provide the clinical practitioner with a high degree of flexibility in bringing the apparatus in close proximity to the region of interest.

It should be understood that the term “coil” includes the use of wires, printed traces or conductive paths, and materials included in the coils that may be temporarily or permanently magnetized.

In accordance with disclosed embodiments, pulsed magnetic fields from the nerve-affecting coils may induce electric fields that inhibit transmission and/or initiation of nervous signals.

The exact mechanism through which pulsed magnetic fields can reduce or increase pain and/or nerve signal transmission is not fully understood, as pointed out by DeSantana. The pain-affecting effect may be due to the triggering of refractory periods in stimulatory nerves, the stimulation of inhibitory or excitatory nerves, or interference with transmission of nerve signals. However, regardless of the exact cause, it is widely appreciated that nerves operate using electrical signals, and that changing magnetic fields can affect those signals.

The image data indicative of images produced with the imaging coils can be collected with magnetic resonance imaging, using either a pulsed or static magnetic field to align the spins of interest, and then using radiofrequency and/or magnetic gradient pulses to re-align and collect information from the spins of interest. The term “spins of interest” is intended to include protons, electrons, magnetizable particles, or other materials with magnetic moments that can be manipulated with external magnetic fields, for those materials that are located in the region of interest. Some of the image data collected with the imaging coils can be used to measure the magnitude and location of the magnetic pulses generated in the region of interest by the nerve-affecting coils. This can be accomplished, for example, by first polarizing spins in the region of interest with the imaging coils, and then depolarizing some of the spins with the nerve-affecting coils. Image data for subsequent images collected with the imaging coils may demonstrate lower signal in the areas where the magnetic fields caused by the nerve-affecting coils were most intense.

In accordance with at least one disclosed embodiment, one mode of operation of may include use of the imaging coils to select a location within a region of interest in which a test set of magnetic pulses generated by the nerve-affecting coils will have maximal pain-reducing or pain-inducing effect in order to provide diagnostic information. The imaging coils can subsequently verify that the magnetic pulses were indeed maximal over the intended location, as described above (e.g., by reducing polarization of spins in the region of interest).

In accordance with at least one disclosed embodiment, a human subject who is being cared for by a practitioner may be queried as to whether the application of magnetic pulses from the nerve-affecting coils was effective in either increasing or decreasing pain. A response to such a query, in conjunction with other subjective or objective information obtained about the human subject, may be used in order to plan and/or assess interventions aimed at relieving the subject's discomfort.

It should be understood that nerve-affecting coils may be able to permanently affect nerve function, for example, by focusing radiofrequency energy at a location, or by inducing strong currents at a location (e.g., through magnetic field changes), or by concentrating magnetizable particles (e.g., with or without a payload) to a location, or by heating magnetizable particles at a location (e.g., with changing magnetic fields).

It should be understood that the apparatus and associated methodologies provided in accordance with the disclosed embodiments may be used in conjunction with other tools or methods to permanently affect nerve function, for example by directing high-intensity focused ultrasound beams to a location, or by directing an antenna or needle (for radiofrequency energy deposition, or for deposition of a chemical) to a location.

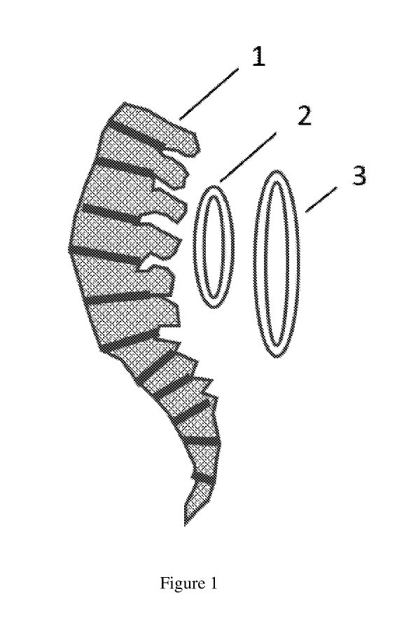

With this understanding of the technical effect and innovation in mind, FIG. 1 provides a schematic of apparatus components utilized in accordance with the disclosed embodiments shown adjacent to the spine 1.

The nerve-affecting coil 2 is illustrated along with imaging coil 3. However, as explained above, one or more of the nerve-affecting coils used in an apparatus provided in accordance with the disclosed embodiments may also be used as one or more of the imaging coils, and vice versa. The imaging and/or nerve-affecting coils may be arrayed in planar and/or single-sided form in order to provide the clinical practitioner with a high degree of flexibility in bringing the apparatus in close proximity to the region of interest. Moreover, these coils may be implemented using wires, printed traces or conductive paths, and materials that may be temporarily or permanently magnetized.

It should be understood that the operations explained herein may be implemented in conjunction with, or under the control of, one or more general purpose computers running software algorithms to provide the presently disclosed functionality and turning those computers into specific purpose computers.

Moreover, those skilled in the art will recognize, upon consideration of the above teachings, that the above exemplary embodiments may be based upon use of one or more programmed processors programmed with a suitable computer program. However, the disclosed embodiments could be implemented using hardware component equivalents such as special purpose hardware and/or dedicated processors. Similarly, general purpose computers, microprocessor based computers, micro-controllers, optical computers, analog computers, dedicated processors, application specific circuits and/or dedicated hard wired logic may be used to construct alternative equivalent embodiments.

Moreover, it should be understood that control and cooperation of components (e.g., magnets) of an instrument for applying magnetic fields described herein to manipulate one or more particles may be provided using software instructions that may be stored in a tangible, non-transitory storage device such as a non-transitory computer readable storage device storing instructions which, when executed on one or more programmed processors, carry out he above-described method operations and resulting functionality. In this case, the term non-transitory is intended to preclude transmitted signals and propagating waves, but not storage devices that are erasable or dependent upon power sources to retain information.

Accordingly, such an instrument may include one or more controllable electromagnetic field sources and a controller that enables control of resulting magnetic fields as described herein. In one such implementation, one or more gradient coils may be utilized under the control of a controller to enables control of the gradient to produce one or magnetic fields using at least one coil driver, wherein one or more coils are provided for transmitting RF energy into a tissue sample of a body part as part of diagnostic, prognostic, and/or treatment

Those skilled in the art will appreciate, upon consideration of the above teachings, that the program operations and processes and associated data used to implement certain of the embodiments described above can be implemented using disc storage as well as other forms of storage devices including, but not limited to non-transitory storage media (where non-transitory is intended only to preclude propagating signals and not signals which are transitory in that they are erased by removal of power or explicit acts of erasure) such as for example Read Only Memory (ROM) devices, Random Access Memory (RAM) devices, network memory devices, optical storage elements, magnetic storage elements, magneto-optical storage elements, flash memory, core memory and/or other equivalent volatile and non-volatile storage technologies without departing from certain embodiments of the present invention. Such alternative storage devices should be considered equivalents.

While certain illustrative embodiments have been described, it is evident that many alternatives, modifications, permutations and variations will become apparent to those skilled in the art in light of the foregoing description. While illustrated embodiments have been outlined above, it is evident that many alternatives, modifications and variations will be apparent to those skilled in the art. Accordingly, the various embodiments of the invention, as set forth above, are intended to be illustrative, not limiting. Various changes may be made without departing from the spirit and scope of the invention.

As a result, it will be apparent for those skilled in the art that the illustrative embodiments described are only examples and that various modifications can be made within the scope of the invention as defined in the appended claims.

We claim:

1. An apparatus for pain assessment, the apparatus comprising:
at least one nerve-affecting coil generating a pulsed magnetic field for affecting a function of at least one innervated pain assembly in a vicinity of the at least one nerve-affecting coil; and
at least one imaging coil generating a magnetic field for collecting image data from a region of interest including the at least one innervated pain assembly,
wherein the pulsed magnetic field from the at least one nerve-affecting coil inhibits transmission and/or initiation of nervous signals.

2. The apparatus of claim 1, wherein the at least one innervated pain assembly includes one or more portions of one or more nerves, nerve roots, ganglia, and/or nerve bundles.

3. The apparatus of claim 2, wherein the at least one nerve-affecting coil produces pulsed magnetic fields that are focused on one or more innervated pain assemblies suspected of causing pain, and wherein the at least one imaging coil produces a pulsed magnetic field for imaging a region that includes the one or more nerve or innervated pain assemblies.

4. The apparatus of claim 1, wherein the at least one nerve-affecting coil is segmented, with sections for selective energizing, de-energizing, or modulation thereby moving a location of maximal magnetic field imposed by the at least one nerve-affecting coil.

5. The apparatus of claim 1, further comprising a plurality of nerve-affecting coils including the at least one nerve-affecting coil, wherein the plurality of nerve-affecting coils are activated independently.

6. The apparatus of claim 1, further comprising one or more electrical supplies with corresponding one or more control modulation circuits coupled thereto and being coupled to and powering the at least one nerve-affecting coil.

7. The apparatus of claim 1, wherein the at least one imaging coil is one of a radiofrequency coils for transmission and/or receipt of radiofrequency energy, a magnetic polarization and/or pre-polarization coil, and a magnetic gradient coil.

8. The apparatus of claim 7, wherein the at least one imaging coil and the at least one nerve-affecting coil are arrayed in planar and/or single-sided form.

9. The apparatus of claim 1, wherein image data indicative of images produced with at least one imaging coil is collected with magnetic resonance imaging without moving the body part.

10. The apparatus of claim 9, with the collection of image data obtained using either a pulsed or static magnetic field to align spins of interest, and then using radiofrequency and/or magnetic gradient pulses to re-align and collect information from the spins of interest.

11. The apparatus of claim 9, wherein at least some of the image data collected is used to measure a magnitude and location of magnetic pulses generated in the region of interest by the at least one nerve-affecting coil.

12. The apparatus of claim 11, wherein measurement of the magnitude and location of magnetic pulses is performed by first polarizing spins in the region of interest with the at least one imaging coil, and then depolarizing at least some of the spins with the at least one nerve-affecting coil.

13. The apparatus of claim 1, wherein a magnetic field distribution created by the at least one nerve-affecting coil is measured by collection of image data with the at least one imaging coil.

14. The apparatus of claim 1, wherein a magnetic field distribution created by the at least one nerve-affecting coil is measured by analyzing image data with the at least one imaging coil.

15. A method for assessing pain, the method comprising:
generating, using at least one nerve-affecting coil, a pulsed magnetic field for affecting a function of at least one innervated pain assembly in a vicinity of the at least one nerve-affecting coil; and
generating, using at least one imaging coil, a magnetic field for collecting image data from a region of interest including the at least one innervated pain assembly,
wherein the pulsed magnetic field from the at least one nerve-affecting coil inhibits transmission and/or initiation of nervous signals.

16. The method of claim 15, wherein the at least one innervated pain assembly includes one or more portions of one or more nerves, nerve roots, ganglia, and/or nerve bundles.

17. The method of claim 16, wherein the at least one nerve-affecting coil produces pulsed magnetic fields that are focused on one or more innervated pain assemblies suspected of causing pain, and wherein the at least one imaging coil produces a pulsed magnetic field for imaging a region that includes the one or more nerve or innervated pain assemblies.

18. The method of claim 15, wherein the at least one nerve-affecting coil is segmented, with sections for selective energizing, de-energizing, or modulation thereby moving a location of maximal magnetic field imposed by the at least one nerve-affecting coil.

19. The method of claim 15, further independently activating a plurality of nerve-affecting coils including the at least one nerve-affecting coil.

20. The method of claim 15, further comprising powering the at least one nerve-affecting coil using one or more electrical supplies with corresponding one or more control modulation circuits coupled thereto and being coupled to the at least one nerve-affecting coil. The method of claim 15, further comprising selecting an appropriate target area for intervention based at least on the collected image data from the region of interest including the at least one innervated pain assembly.

21. The method of claim 15, wherein image data indicative of images produced with at least one imaging coil is collected with magnetic resonance imaging without moving the body part.

22. The method of claim 15, wherein image data indicative of images produced with at least one imaging coil is collected with magnetic resonance imaging using either a pulsed or static magnetic field to align spins of interest, and then using radiofrequency and/or magnetic gradient pulses to re-align and collect information from the spins of interest.

23. The method of claim 22, wherein at least some of the image data collected is used to measure a magnitude and location of magnetic pulses generated in the region of interest by the at least one nerve-affecting coil.

24. The method of claim 23, wherein measurement of the magnitude and location of magnetic pulses is performed by first polarizing spins in the region of interest with the at least one imaging coil, and then depolarizing at least some of the spins with the at least one nerve-affecting coil.

25. The method of claim 15, wherein a magnetic field distribution created by the at least one nerve-affecting coil is measured by collection of image data with the at least one imaging coil.

26. The method of claim 15, wherein a magnetic field distribution created by the at least one nerve-affecting coil is measured by analyzing image data with the at least one imaging coil.

27. The method of claim 15, further comprising querying a subject as to a sensation experienced during or after application of a magnetic field by the at least one nerve-affecting coil to predict efficacy of an interventional procedure.
